(19) United States
(12) Patent Application Publication
(10) Pub. No.: US 2017/0238804 A1
(43) Pub. Date: Aug. 24, 2017

(54) SYSTEMS FOR CONTINUOUS AND NON-CONTINUOUS IN-VIVO SPECTROSCOPY AND METHODS THEREFOR

(71) Applicant: Memiray Inc., Arcadia, CA (US)

(72) Inventor: Frank Thomas Hartley, Arcadia, CA (US)

(21) Appl. No.: 15/430,915

(22) Filed: Feb. 13, 2017

Related U.S. Application Data
(60) Provisional application No. 62/298,046, filed on Feb. 22, 2016.

(51) Int. Cl. A61B 5/00

(52) U.S. Cl. CPC A61B 5/0075; A61B 5/002; A61B 2562/0242

(57) ABSTRACT

Spectroscopy systems suitable for estimating the composition of test samples are disclosed. Embodiments of the present invention include an element that can be embedded within a sample and operatively couple with elements of the system located outside the sample, thereby enabling long-term monitoring of the sample. An embodiment includes radiation-emitting and radiation-detecting devices having periodic structures, such as photonic crystals and/or plasmonic metamaterials, which serve to filter the wavelengths of radiation at which they operate and/or enhance responsivity for those wavelengths. In some embodiments, the detecting devices are housed in a module suitable for long-term implantation within the sample. In some embodiments, the radiation-emitting and detecting devices are located external to the sample and are optically coupled with a mirror implanted within the sample. In some embodiments, an estimate of the composition of the test sample is generated at controller that is in communication with the emitter module.

### RELATED APPLICATIONS

This application claims the benefit of U.S. Provisional Application No. 62/298,046 filed Feb. 22, 2016 (Attorney Docket: 550-004PR3). The entire disclosure of U.S. Provisional Application No. 62/298,046 is incorporated herein by reference.

### FIELD OF THE INVENTION

The present invention relates to systems and methods for continuous and non-continuous in-vivo spectroscopy and, more particularly, to mid-infrared spectroscopy.

### BACKGROUND OF THE INVENTION

Infrared spectroscopy is a well-known technique for analyzing the chemical makeup of a sample. Spectroscopy systems (i.e., spectrometers) are widely used in many applications, such as medical diagnostics, petroleum exploration, environmental health monitoring, and drug testing.

Infrared spectroscopy exploits the fact that every chemical selectively absorbs radiation at a set of wavelengths that is characteristic of that chemical. During infrared spectroscopy, infrared radiation is directed at a sample and detected after it has interacted with the sample. The input signal and output signal are compared to determine the positions, magnitudes, and inflections of spectral peaks in the output signal imparted by absorption in the sample, thereby providing a “spectral fingerprint” that is indicative of its chemical composition.

Infrared spectroscopy typically employs radiation in the near-infrared (NIR) spectral range (i.e., radiation having wavelengths within the range of approximately 0.7 microns to 2.5 microns) or radiation in the mid-infrared (MIR) spectral range (i.e., radiation having wavelengths within the range of approximately 2.5 microns to 12.5 microns). The MIR spectral range is particularly well suited for use in spectroscopy because it represents an information-rich spectral region for most chemicals, while the NIR spectral range provides less chemical-signature information. For these reasons, the use of MIR radiation is preferable to the use of NIR radiation.

Unfortunately, many samples include a background chemical (i.e., solvent) that is highly absorptive of MIR radiation, while being less absorptive for NIR radiation. For example, in blood analysis, brain scanning, and the like, water makes up a significant portion of the biological tissue being analyzed (approximately 80% of blood is water). Water has a very high absorption coefficient over much of the MIR spectral range.

As a result, in order to realize an output signal having sufficient signal-to-noise ratio for reliable detection, the path length through a biological sample must be kept extremely short or a very intense MIR radiation signal must be used to interrogate the sample. In medical applications, therefore, MIR spectroscopy has been primarily used for ex-vivo analysis of tissue samples that can be sectioned into very thin pieces or liquid samples that can be squeezed between microscope slides. In those cases where MIR spectroscopy has been used for in-vivo analysis, it has typically been relegated to analysis of very thin tissue regions, such as the webbing between fingers, the ear lobe, the eyelid, and the like.

In addition, for many in-vivo medical applications, exposure to high-power radiation signals for extended durations can lead to patient discomfort and/or tissue damage. As a result, continuous or quasi-continuous MIR spectroscopy over an extended duration is generally not practiced. Furthermore, the size and expense of infrared spectroscopy systems precludes their being dedicated to a single procedure for an extended duration.

A system that enables high-performance MIR spectroscopy, with the potential for quasi-continuous or continuous operation and/or for extended time periods, would be a significant advance in the state of the art.

### SUMMARY OF THE INVENTION

The present invention enables MIR spectroscopy systems that overcome some of the costs and disadvantages of the prior art. Embodiments of the present invention enable spectroscopic monitoring of a sample over an extended period of time by implanting a first portion of an MIR spectroscopy system within the sample and interfacing with it via a second portion that is located external to the sample. Furthermore, embodiments of the present invention include high-sensitivity thermal detectors that enable high-signal-to-noise performance with less intense interrogating radiation than required in the prior art. Embodiments of the present invention are particularly well suited for use in medical applications, such as non-invasive blood and/or tissue analysis, and extended-duration in-vivo medical monitoring and diagnostic procedures.

Embodiments of the present invention employ radiation sources that provide radiation signals sufficient to enable spectroscopy through water-containing samples with good performance. In addition, embodiments of the present invention comprise detectors that can selectively detect radiation within narrow wavelength ranges, thereby reducing noise in their output signals. By forming an array of such detectors, each detecting a different wavelength range, a spectral signature for a chemical, compound, environment, etc., can be determined.

An illustrative embodiment of the present invention is an MIR spectroscopy system comprising an emitter module, a detector module, and a controller, where the detector module is dimensioned and arranged for implantation within a test sample, such as a human body, while the emitter module remains outside the sample. The emitter module includes a plasmonic broadband emitter, a communications module, and a processor for controlling spectroscopic measurements. The detector module includes an array of MIR thermal detectors and a communications module for communicating with the emitter module. An alignment system locates the detector module relative to the emitter module such that they are optically coupled when the emitter module is placed in its operating position against the sample.

The source provides radiation that is substantially confined to the MIR spectral range. Within the MIR spectral range, its output has an intensity that is three orders of magnitude higher than out of the MIR spectral range. The source is a plasmonic-metamaterial-based element that comprises a thin-film platinum filament having a serpentine shape, which is overcoated with a layer of dielectric material comprising sapphire. Patterns of isolated platinum features are disposed on the top surface of the sapphire layer, where the patterns effect resonances within the MIR spectral range. The resonances enhance emission within this range while suppressing emissions outside it.

The detection module includes an array of detector elements, each of which includes a substantially identical thermal detector that is thermally coupled with an absorber comprising a photonic crystal. The photonic crystal of each detector element is dimensioned and arranged such that its respective absorber selectively absorbs a different narrow range of wavelength within the MIR spectral range. Upon the selective absorption of radiation, each absorber heats up commensurately, and its temperature change is detected by its corresponding thermal detector. By providing the detector elements with high selectivity for their respective narrow detection band, the signal-to-noise ratio of the detected signal is improved.

The output of the detector is provided to the emitter module via a low-power, short-range RF link comprising elements of the alignment system. The emitter module performs some pre-processing of the output signal and provides it to the controller via a second RF communications link. The controller employs a computer application to generate an estimate of the test sample based on the provided data.

In some embodiments, the emitter module and detector module communicate via an acoustic communications link. In some embodiments, the emitter module and the controller communicate via an acoustic communications link.

In some embodiments, the source includes a filament made of a metal other than platinum and/or a pattern other than serpentine. In some embodiments, the dielectric layer disposed on the filament comprises a dielectric other than sapphire. In some embodiments, the resonant pattern comprises a metal other than platinum.

An embodiment of the present invention is a spectroscopy system comprising: (1) an emitter module comprising a source for providing a first radiation signal, the source including; (a) a filament comprising a first material that is electrically conductive; (b) a first layer disposed on the filament, the first layer comprising a dielectric material; and (c) second layer disposed on the first layer, the second layer comprising a plurality of features that is arranged in a periodic arrangement; wherein the filament, first layer and second layer collectively define a plasmonic resonant structure that is operative for providing first radiation signal such that it that has higher intensity within a first spectral range than outside the first spectral range; and (2) a detector module comprising a detector that includes a plurality of detector elements, each detector element being selectively sensitive for a different first sub-range of wavelengths within the first spectral range; wherein, when operatively coupled, the source and detector collectively define an optical path that includes at least a portion of a test sample.

Another embodiment of the present invention is a method for analyzing a test sample, the method comprising: providing an emitter module comprising a source that includes a plasmonic resonant structure; energizing the source to generate a first radiation signal that has higher intensity within a first spectral range than outside the first spectral range, wherein the first spectral range is within the mid-infrared spectral range; transmitting the first radiation signal through a first portion of the test sample; and estimating the composition of the test sample based on absorption of the first radiation signal in first portion.

### BRIEF DESCRIPTION OF THE DRAWINGS

FIG. 1 depicts a schematic drawing of a spectroscopy system in accordance with an illustrative embodiment of the present invention.

FIG. 2A depicts a schematic drawing of a side view of alignment system 106 in an operative arrangement.

FIG. 2B depicts a schematic drawing of a top view of an alignment feature in accordance with the illustrative embodiment of the present invention.

FIGS. 3A-B depict schematic diagrams of top and cross-sectional views, respectively, of a radiation source in accordance with the illustrative embodiment.

FIG. 4A depicts a schematic drawing of a cross-sectional view of a detector module in accordance with the illustrative embodiment.

FIG. 4B depicts a schematic drawing of a representative detector element in accordance with the illustrative embodiment.

FIG. 5 depicts operations of a method suitable for analyzing a test sample in accordance with the illustrative embodiment.

FIG. 6 depicts a radiation source in accordance with an alternative embodiment of the present invention.

FIG. 7 depicts a spectroscopy system in accordance with an alternative embodiment of the present invention.

### DETAILED DESCRIPTION

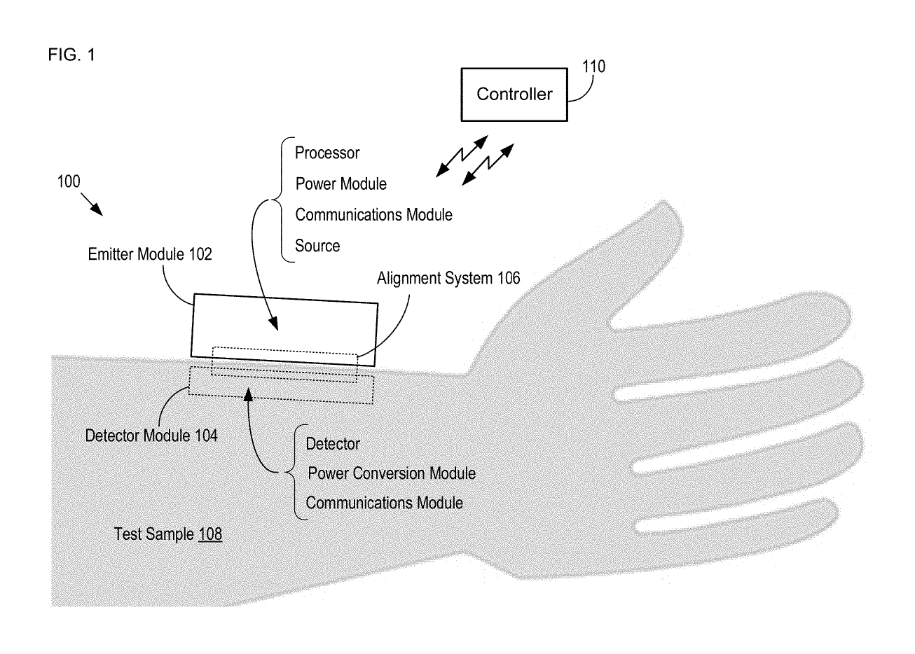

FIG. 1 depicts a schematic drawing of a spectroscopy system in accordance with an illustrative embodiment of the present invention. System 100 is an MIR spectroscopy system suitable for extended-duration in-vivo spectroscopy of a tissue region in a human patient. System 100 includes emitter module 102, detector module 104, alignment system 106, and controller 110.

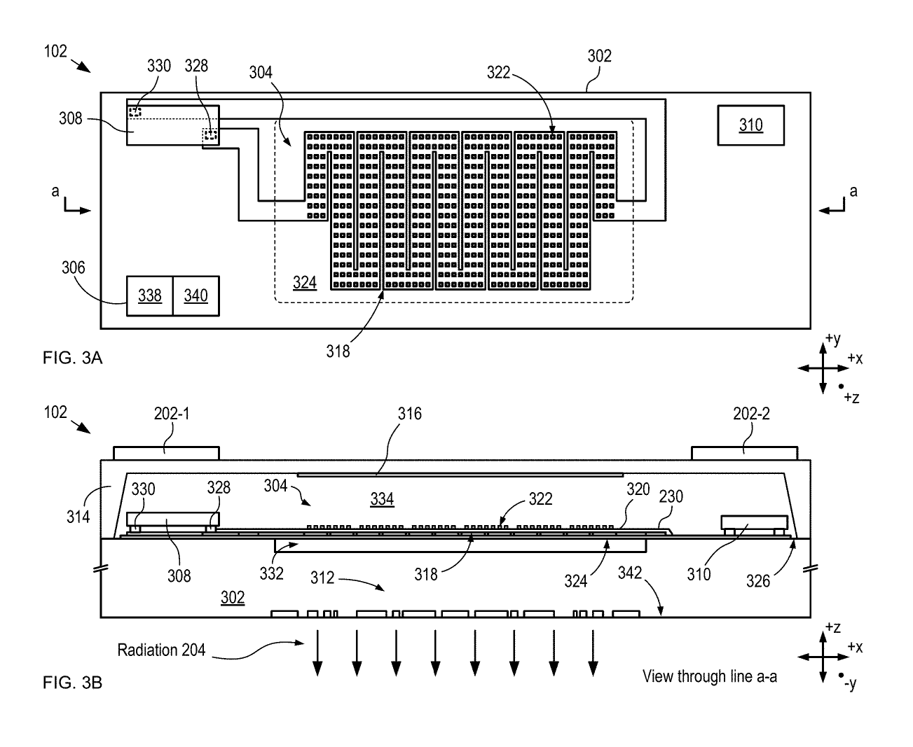

Emitter module 102 includes an MIR radiation source, as well as additional modules that enable it to be operatively coupled with the detector module while the detector module is embedded within test sample 108 and the emitter module remains outside it. Typically, emitter module 102 includes modules that enable communications with detector module 104 and other equipment and/or personnel, wireless powering of detector module 104, data processing, and the like. Emitter module 102 is described in detail below and with respect to FIGS. 3A-B.

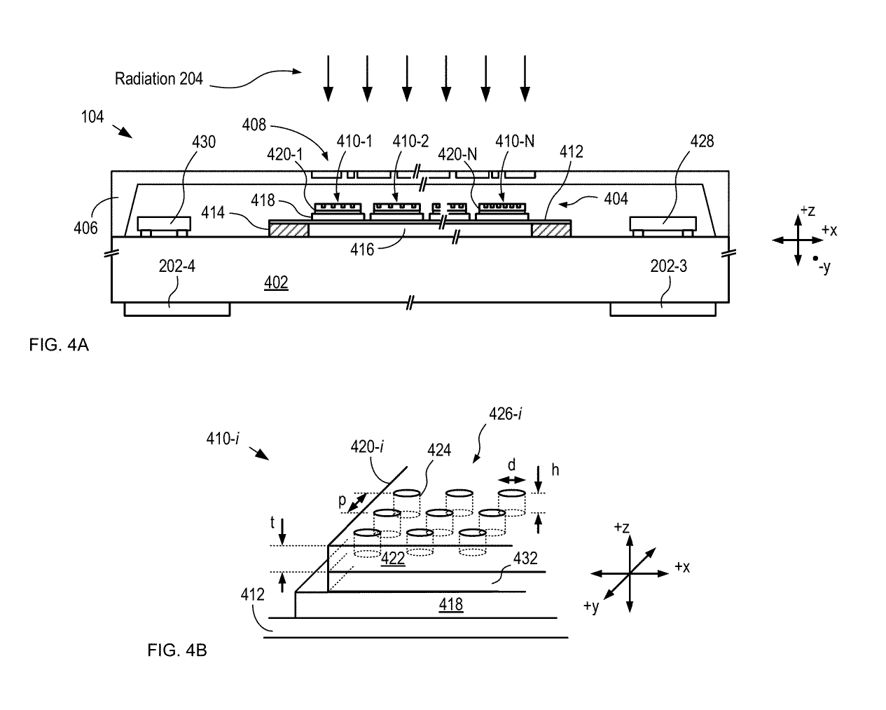

Detector module 104 is an MIR radiation detection module that is designed for implantation within test sample 108. Detector module 104 includes an MIR detector, a power conversion module for converting power provided by emitter module 102 into useable electrical energy, and a communications module that enables communication with emitter module 102. Detector module 104 is described in detail below and with respect to FIG. 4.

For proper operation, the emitter and detector modules must be aligned such that an optical path is established between the MIR source on the emitter module and the MIR detector on the detector module. Alignment system 106 enables proper alignment of the modules. In some embodiments, including the depicted example, alignment system 106 also serves as a wireless power channel that enables the detector module to be powered while it is located within test sample 108. Further, in some embodiments, including the depicted example, alignment system 106 also operates as a short-range RF communications link between the emitter and detector modules.

Controller 110 is a system operative for communicating with emitter module 102, analyzing the spectral characteristics of test sample 108 to estimate its composition, and communicating with external equipment. In the depicted example, controller 110 is a conventional smart phone running an application for performing spectroscopic analysis of a test sample. In some embodiments, controller 110 is an alternative processing system, such as a base station, personal computer, etc.

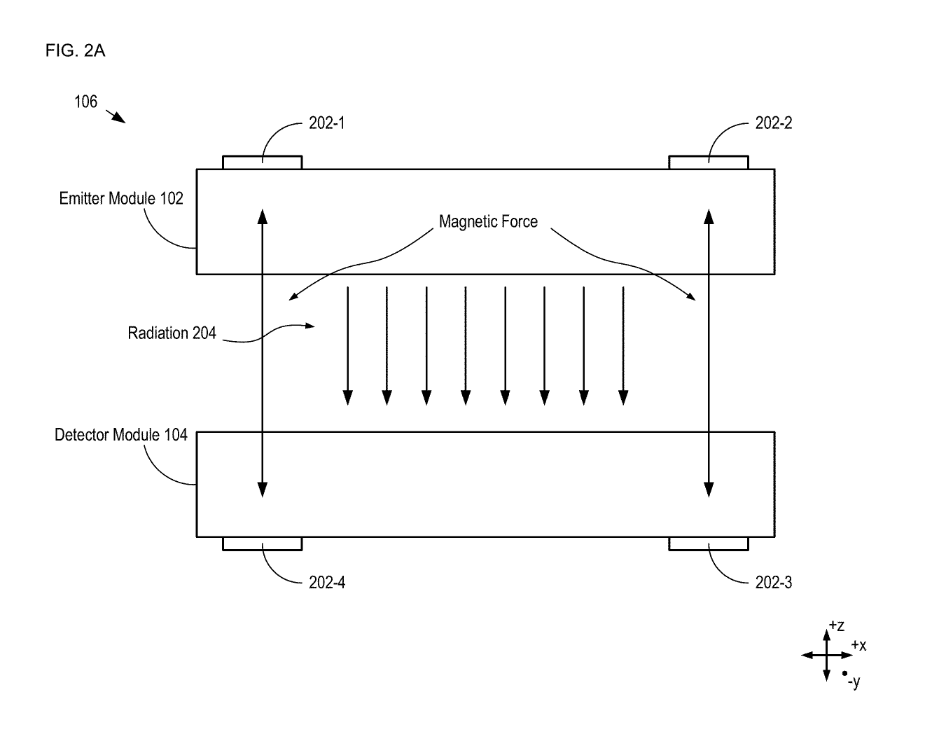

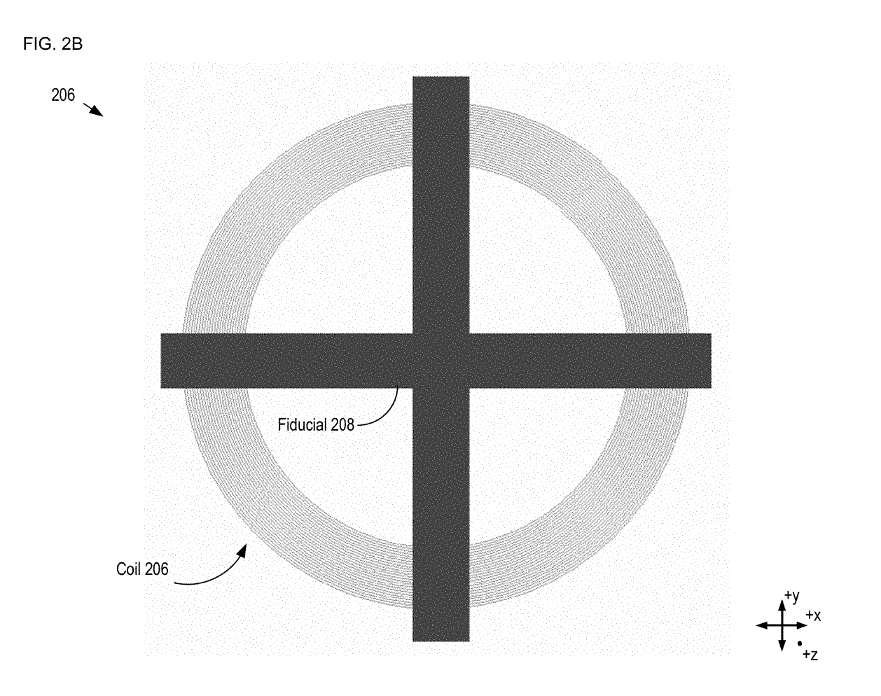

FIG. 2A depicts a schematic drawing of a side view of alignment system 106 in an operative arrangement. Alignment system 106 includes alignment features 202-1 through 202-4, where alignment features 202-1 and 202-2 are incorporated in emitter module 102 and alignment features 202-3 and 202-4 are incorporated in detector module 104. The alignment features operate cooperatively to generate a magnetic force that aligns the emitter and detector modules to establish an optical path for radiation 204 from the MIR source on emitter module 102 and the MIR detector on detector module 104.

FIG. 2B depicts a schematic drawing of a top view of an alignment feature in accordance with the illustrative embodiment of the present invention. Alignment feature 202 includes coil 206 and fiducial 208.

Coil 206 is a planar coil of electrically conductive material. In the depicted example, coil 206 comprises platinum; however, any conductive material can be used for coil 206 without departing from the scope of the present invention.

Fiducial 208 is a “cross-shaped” pattern of a “soft” magnetic material, such as iron, low-carbon steel, iron-silicon alloy, iron-aluminum-silicon alloy, nickel-iron alloy (e.g., Permalloy, etc.), iron-cobalt alloy, ferrites, and the like.

Fiducial 208 is aligned with coil 206 such that it is magnetized by a magnetic field generated by current flow through the coil.

When a direct-current (DC) electric current flows through the actively driven coils of each of alignment features 202-1 and 202-2, the coils generate magnetic fields that give rise to current flows in the passive coils of alignment features 202-3 and 202-4, which generates magnetic fields at each of these passive coils. The magnetic fields generated by each active and passive coil couples into and magnetizes its respective fiducial 208. The magnetized fiducials operate as multipole alignment facilities, which give rise to a multipole DC magnetic circuit that produces an attractive force between the emitter and detector modules. This force aligns them laterally and rotationally while also reducing the separation between them. In some embodiments, each of emitter module 102 and detector module 104 includes more than two alignment features 202.

FIGS. 3A-B depict schematic diagrams of top and cross-sectional views, respectively, of a radiation source in accordance with the illustrative embodiment. Emitter module 102 comprises substrate 302, source 304, communications module 306, power system 308, processor 310, lens 312, cap 314, reflector 316, and alignment features 202-1 and 202-2. It should be noted that, for clarity, cap 314, reflector 316, and alignment features 202-1 and 202-2 are not shown in FIG. 2A.

Substrate 302 is a conventional substrate suitable for use for planar processing. In the depicted example, substrate 302 is made of float-zone silicon and has a thickness of approximately 500 microns; however, one skilled in the art will recognize, after reading this Specification, that any material that is substantially transparent for MIR radiation is suitable for use in substrate 302 and that the substrate can have any practical thickness.

Source 304 comprises filament 318, dielectric layer 320, and nanodots 322, which are disposed on suspended membrane 324 such that they are collectively suspended above substrate 302. Typically, source 304 is fabricated on substrate 302 using conventional Micro Electro Mechanical Systems (MEMS) fabrication techniques, including layer deposition, etching, lift-off patterning, subtractive patterning, etc. It will be clear to one skilled in the art, after reading this Specification, how to fabricate source 304.

Membrane 324 is a layer of structural material disposed on surface 326 of substrate 302. In the depicted example, membrane 324 comprises silicon-rich silicon nitride; however, one skilled in the art will recognize that many other structural materials (e.g., polysilicon, single-crystal silicon, stoichiometric silicon nitride, silicon oxynitride, silicon carbide, compound semiconductors, germanium, alumina, etc.) can be used without departing from the scope of the present invention.

Filament 318 is a “serpentine-shaped” platinum trace disposed on membrane 324. Filament 318 has a thickness within the range of approximately 20 nm to approximately 1 micron, a total length that is within the range of approximately 100 microns to approximately 10 mm, and a width within the range of approximately 500 nm to approximately 250 microns. Typically, filament 318 is formed by depositing a full-surface film of platinum and subsequently patterning it using conventional photolithography and etching methods to define its shape; however, in some embodiments, filament 318 is formed using conventional “lift-off” techniques.

Although the illustrative embodiment comprises a platinum filament having a serpentine shape, it will be clear to one skilled in the art, after reading this Specification, how to specify, make, and use alternative embodiments of the present invention wherein filament 318 has a shape other than serpentine and/or comprises a material other than platinum (e.g., tungsten, titanium-tungsten, etc.).

Dielectric layer 320 is a layer of sapphire that overcoats filament 318. Dielectric layer 320 typically has a thickness within the range of approximately 10 nm to approximately 2 microns. One skilled in the art will recognize, after reading this Specification, that other dielectric materials can be used in dielectric layer 320 without departing from the scope of the present invention. Examples of other materials suitable for use in dielectric layer 320 include, without limitation, silicon oxides, silicon nitrides, silicon oxynitride, glasses, other oxides, other nitrides, and the like.

Nanodots 322 are regions of platinum disposed on dielectric layer 320 to collectively define a resonant pattern for radiation within a desired spectral band. Each nanodot 322 has a cross-sectional shape that is substantially a square having sides within the range of approximately 5 nm to approximately 2000 nm. Nanodots 322 are arranged in a periodic arrangement having a period within the range of approximately 500 nm to approximately 5 microns. In some embodiments, at least some of nanodots 322 have a cross-sectional shape of another size and/or a shape other than square, such as a rectangle, circle, ellipse, polygon, or irregular (e.g., cross, c-shape, h-shape, triton, chevron, etc.). It should be noted that, in some embodiments, the nanodots themselves have internal resonances that affect the overall spectral characteristics of source 304.

In some embodiments, the shape of each of nanodots 322 is etched into the surface of dielectric layer 320 thereby transferring the nanodot pattern into the dielectric layer itself. It should be noted that the depth to which the pattern extends into the dielectric layer, as well as the dielectric constant of dielectric layer 320 and the material of nanodots 322, can be controlled to affect the emission characteristics (e.g., spectral characteristics, etc.) of source 304.

Further, in some embodiments, nanodots are formed such that they are quantum dots or quantum-dot phosphors, thereby providing additional control over the emissive properties of source 304.

Filament 318 is electrically connected to power system 308 via electrical contacts 328 and 330, which also provide die-attach bonding sites for the integrated-circuit die containing power system 308.

Membrane 324 is separated from 302 by cavity 332. As a result, filament 318 and substrate 302 are substantially thermally decoupled. This mitigates leakage of thermal energy from the filament into the substrate, thereby improving the efficiency of source 304. In addition, because the filament and membrane have very low thermal mass, filament 318 can respond more quickly when current flow through it is initiated and stopped.

It should be noted that absorption of MIR radiation in a water-containing test sample can lead to heating in the test sample that can damage the tissue of the sample. While this might be acceptable for some ex-vivo measurements, in-vivo material could be heated to a degree where pain and tissue damage manifest. As a result, the FDA imposes strict limitations on any form of radiation directed into a patient.

Because it is low thermal mass, filament 318 can be operated in a “flash” mode, in which it is driven with a periodic drive signal comprising a series of drive pulses. As a result, the filament emits radiation 204 as a series of radiation pulses to keep the integrated radiation exposure of the test sample below its damage threshold. Such operation mitigates tissue damage in test sample 108 from overexposure to radiation 202. In some cases, it also allows the radiation pulses to have higher intensity than would otherwise be permissible, thereby enabling improved system performance. Preferably, the duty factor of the periodic drive signal is below 50%.

In some embodiments, membrane 324 is formed on a sacrificial layer disposed on surface 326 and a portion of the sacrificial layer is subsequently removed to form cavity 332. In some embodiments, filament 318 is formed on substrate 302 without a cavity and the filament and substrate are separated by one or more thermally insulating layers.

It is another aspect of the present invention that filament 318, dielectric layer 320, and nanodots 322 collectively define a plasmonic metamaterial that defines a plasmonic resonant structure (i.e., a plasmon polariton resonator). As a result, source 304 emits radiation that has enhanced intensity within a specific radiation spectrum—specifically, the MIR spectral range.

The term “plasmonics” refers to the interactions between an electromagnetic field and free electrons in a metal that can be excited by the electric component of radiation to have collective oscillations. Ordinarily, ohmic loss and electron-core interactions are detrimental to plasmonic oscillation. Through proper design of a metal pattern for surface plasmonic excitation, however, the absorption of radiation in the metal can be enhanced affording embodiments of the present invention advantages over plasmonic devices of the prior art.

The present invention comprises a narrow-band source that is based on a novel plasmonics effect wherein the electric component of the collective oscillations of emitted radiation is absorbed in metal patterns formed on a dielectric radiator. Surface plasmon polariton infrared electromagnetic waves are induced into and travel along the metal-dielectric and metal-air interface and radiate the polariton electromagnetic waves into free space. Through judicious device design, the emission can be enhanced within a specific, desired wavelength range at the expense of energy at wavelengths outside this range, thereby resulting in a radiation source that substantially selectively emits radiation having an extremely high intensity within the desired spectral band.

Although the plasmonic effect is exploited within the context of a radiation source, it will be clear to one skilled in the art, after reading this Specification, that the principles described can also enable a radiation absorber that selectively absorbs amplified narrow-band energy from free space within a desired wavelength range. In addition, in some embodiments, plasmon polariton resonators in accordance with the present invention can provide more efficient and higher flux radiation than prior-art heat exchangers.

Plasmonic metamaterials exploit surface plasmons, which are produced from the interaction of radiation with metal dielectrics. Under specific conditions, the incident radiation couples with the surface plasmons to create self-sustaining, propagating electromagnetic waves known as surface plasmon polaritons.

Surface plasmon polaritons are infrared, visible or terahertz electromagnetic waves, which travel along a metal-dielectric or metal-air interface where the wave involves both charge motion in the metal (“surface plasmon”) and electromagnetic waves in the air or dielectric (“polariton”). Surface plasmon polaritons are shorter in wavelength than the incident radiation (photons) and have tighter spatial confinement and thus higher local field intensity—what we are exploiting. A surface plasmon polariton will propagate along the interface until its energy is lost either to absorption in the metal or scattered into free space.

One skilled in the art will recognize, after reading this Specification, that the size and arrangement of nanodots 322 depend on the wavelength or wavelengths of desired operation for source 304. The size and arrangement of nanodots 322 is designed to establish a resonance condition for the range of frequency photons within the mid-infrared radiation spectrum (i.e., wavelengths within the range of approximately 2.5 micron to approximately 12.5 microns). In this frequency range, the photon-frequency matches the natural frequency of surface electrons that oscillate against the restoring force of positive nuclei within the material of the nanodots, thereby giving rise to localized surface plasmon resonance and surface electromagnetic waves referred to as surface plasmon polaritons.

In the illustrative embodiment, for example, nanodots 322 are arranged in a broadband resonant pattern that, when electric current flows through filament 318, produces markedly increased radiation intensity over the 2.5 micron to 12.5 micron (i.e., the MIR spectrum) bandwidth of interest. The increased energy radiated within this spectral range comes from both the higher frequency radiation and the lower frequency radiation of the strip filament's black body emissions. By “stealing” energy from the higher and lower frequency ranges, in addition to enhancing emission in the desired spectral band (i.e., in-band radiation), the intensity in the higher and lower ranges (i.e., out-of-band radiation) is also reduced. It should be noted that the in-band radiative flux of a plasmonic system in accordance with the present invention can theoretically be greater than the equivalent band blackbody flux from the surface of the sun. It should be noted that sources in accordance with the present invention provide emission spectra that are substantially insensitive to the temperature of the filament itself. This is in contrast to typical responsivity of a conventional black-body source.

Although source 304 is designed to emit radiation within the infrared spectrum, it will be clear to one skilled in the art, after reading this Specification, how to specify, make, and use alternative embodiments of the present invention that emit radiation in another wavelength range. For example, embodiments of the present invention can be designed to emit radiation anywhere on the visible, infrared or terahertz electromagnetic spectrum or scatter narrow-band enhanced energy waves into free space.

Communications module 306, power system 308, and processor 310 are integrated-circuits formed on separate die added to emitter module 102 via conventional multi-chip module packaging techniques. In some embodiments, one or more of these circuits are formed in/on substrate 302 itself.

Communications module 306 includes communications circuits 338 and 340.

Communications circuit 338 is a conventional Bluetooth module for communicating with controller 110. In some embodiments, communications circuit 338 enables RF communications via a different standard protocol (e.g., WiFi, ZigBee, cellular, RFID, etc.).

Communications circuit 340 is a low-power RF communications circuit that communicates with detector module 104 via alignment system 106, as discussed below.

Power system 308 is a multi-functional integrated circuit that is operative for: (1) driving electrical current through filament 318 to generate MIR radiation at source 304; and (2) remotely powering detector module 104 via alignment system 106.

Processor 310 is a conventional microprocessor that is operative for managing the operations necessary for performing spectroscopic analysis of a test sample, such as initiating current flow through filament 318 to generate radiation 204, powering detector module 104, controlling communications between emitter module 102 and detector module 104, compiling the data provided by detector module 104, etc.

Lens 312 is a diffractive lens formed in surface 342 of substrate 302. Lens 312 is operative for spatially and spectrally dispersing the radiation emitted by source 304 and directing the radiation toward detector module 104. Although lens 312 is a diffractive lens, one skilled in the art will recognize, after reading this Specification, that lens 312 can be a refractive lens or a plurality of diffractive and/or refractive lenses. In some embodiments, lens 312 is formed on a surface of cap 314.

Cap 314 is a bulk substrate comprising a suitable structural material (e.g., silicon, alumina, etc.) that includes cavity 334. Cap 314 includes reflector 316, which is disposed on its interior surface. Reflector 316 is a layer of material that is operative for reflecting MIR radiation generated by filament 304 toward detector module 104 during operation of system 100. In some embodiments, reflector 316 is not included in cap 314.

Alignment features 202-1 and 202-2 are disposed on the outer surface of cap 314. In some embodiments, alignment features 202-1 and 202-2 reside on surface 324 of substrate 302. It should be noted that alignment features 202-1 and 202-2 are electrically connected to power system 308 via conventional through-wafer vias and electrical traces; however, for clarity, these are not shown in FIGS. 3A-B.

FIG. 4A depicts a schematic drawing of a cross-sectional view of a detector module in accordance with the illustrative embodiment. Detector module 104 includes substrate 402, detector 404, cap 406, lens 408, power conversion module 428, communications module 430, and alignment features 202-3 and 202-4.

Substrate 402 is analogous to substrate 302 described above.

Detector 404 includes detector elements 410-1 through 410-N, where N is the number of detector elements in the detector. Each of detector elements 410-1 through 410-N (referred to, collectively, as elements 410) is a spectrally selective, narrow-resonance absorber that is sensitive to radiation in a different sub-range of wavelengths within the MIR spectral range. In the depicted example, the spectral width of each sub-range is approximately 1/N of the entire spectral range of radiation signal 204. In the illustrative embodiment, detector 404 includes an 80×60 array of detector elements, each of which is approximately 17 microns×17 microns. As a result, detector 404 has a relatively small footprint (1.4 mm×1 mm) but contains 4800 active detector pixels. In some embodiments, detector 404 is a hyperspectral array having 4800 hyperspectral partitions across the array for a per-pixel bandwidth of approximately 2 nanometers.

Elements 410 are held above substrate 402 by membrane 412, which is separated from substrate 402 by spacer layer 414. Cavity 416 is formed between membrane 412 and substrate 402 by etching away the region of the spacer layer lying under elements 410 after their formation. In some embodiments, elements 410 are formed on membrane 412 after the formation of at least a portion of cavity 416.

FIG. 4B depicts a schematic drawing of a representative detector element in accordance with the illustrative embodiment. Each detector element 410-i (for i=1 through N) includes thermal detector 418 and absorber 420-i, where the absorber is disposed on reflector 432, which thermally couples the absorber with the thermal detector while reflecting light not absorbed by the absorber away from thermal detector 418.

Each of thermal detectors 418 in detector 404 is a substantially identical vanadium-oxide microbolometer. In some embodiments, at least one of thermal detectors 418 comprises a different thermally sensitive element. By virtue of their suspension above the substrate via membrane 412, thermal detectors 418 are substantially thermally isolated from the substrate.

Absorber 420-i includes layer 422 and an arrangement of features 424 that defines photonic crystal 426-i.

Layer 422 is a region of dielectric material having a thickness, t, where the dielectric material is partially transparent for radiation 204. In the depicted example, layer 422 comprises sapphire (refractive index, n,=1.77); however, myriad alternative materials can be used in layer 422 without departing from the scope of the present invention. Materials suitable for use in layer 422 include, without limitation, silicon nitride, silicon oxynitrides, silicon carbides, titanium oxide, silicon dioxide, ceramics, composite materials, semiconductors, and the like.

It is an aspect of the present invention that enhanced absorption of radiation within a selective wavelength range is enabled by forming photonic crystal 426 in absorber 420. One skilled in the art will recognize that a photonic crystal is a structure whose refractive index is modulated with a period comparable to the wavelength of radiation in the material. In some instances, photonics crystals can provide a bridge that enables sustenance of an electromagnetic wave travelling at the interface between two different materials such as two different dielectric materials.

Photonic crystals enable intriguing negative refraction, strong radiation confinement, and extremely slow radiation. The photonic bandgaps of these 2D-photonic crystals create strong radiation confinement in sub-wavelength sized ultrahigh-quality-factor (Q) cavities. A spectrally selectively tuned photonic-bandgap crystal has a few nanometer wide resonance peak that will be absorbed, and massively amplified, with resonant Qs of 10⁶, from normal incidence radiation.

The energy of the absorbed radiation is dissipated as heat in the photonic crystal. The present invention exploits this attribute of a photonic crystal by forming it such that it is thermally coupled with a thermal transducer. As a result, the sensitivity of the thermal transducer is greatly enhanced for the particular hyperspectral selected bandgap. This wavelength selectivity of absorption enables, for example, hyperspectral arrayed thermal detectors suitable for MIR spectroscopy.

One skilled in the art will recognize that radiation not absorbed in absorber 420 will typically pass through it to thermal detector 418. At least some of this radiation can be absorbed in the thermal detector itself, thereby generating that represents noise in the output signal of the detector element. As a result, to mitigate absorption of this light in the thermal detector, each of detector elements 410 includes reflector 432 between absorber 420-i and thermal detector 418.

Reflector 432 is a layer of material that is sufficiently thick to be substantially reflective for MIR radiation while also enabling the heat of absorber 420-i to pass through to thermal detector 418. Materials suitable for use in reflector 432 include, without limitation, metals (e.g., gold, platinum, etc.), multi-layer dielectric mirrors, and the like.

Each of features 424 is a substantially circular hole having a diameter, d, and a depth, h. In the embodiment shown, the depth of each feature is less than the thickness, t, of layer 422 (i.e., each features 424 is a “blind” hole). In some embodiments, at least one of features 424 has a different shape, such as square, polygonal, rectangular, or irregular.

The size, depth, and spacing, p, of features 424, as well as the material characteristics of layer 422 (e.g., refractive index, etc.), are matters of design and are based on the specific range of wavelengths for which element 410-i is intended to be absorbing. Typically, these features are less than the wavelength of operation. As a result, at least some of these design features are different for each different detector element to enable each to selectively absorb a different range of wavelengths. Each spectrally selectively tuned photonic crystal 426-i typically has a few nanometer-wide resonance peaks that will be absorbed in absorber 420-i, and massively amplified with resonant Qs of 10⁴, from normal incidence radiation. The energy of the absorbed light is converted into thermal energy in the absorber, thereby changing the temperature of its respective thermal detector 418. As a result, the sensitivity of the thermal detector is greatly enhanced for the particular hyperspectral selected bandgap of its respective photonic crystal.

It should be noted that the sensitivity of a thermal detector 418 in accordance with the illustrative embodiment is approximately 10⁹ cm rt(Hz)/W. When coupled with a corresponding photonic crystal 426-i that amplifies with a resonant Q of order 10⁴, the sensitivity to selectively absorbed incidence radiation of each element 410-i is predicted to be of order 10¹⁵ cm rt(Hz)/W—better than even the most exotic cryo-cooled detectors.

In some embodiments, detector 104 is operatively coupled with a wavelength dispersion element (e.g., a prism, diffraction grating, diffractive lens, blazed grating, eschelle grating, etc.) that receives broad spectrum radiation and spatially disperses the wavelengths along elements 410 such that each element 410-i receives a different sub-set of wavelengths.

It should be noted that detector 404 is merely one example of a multi-detector-element device suitable for use in embodiments, of the present invention and that any alternative detector comprising a plurality of spectrally selective detector elements can be used in detector module 104 without departing from the scope of the present invention.

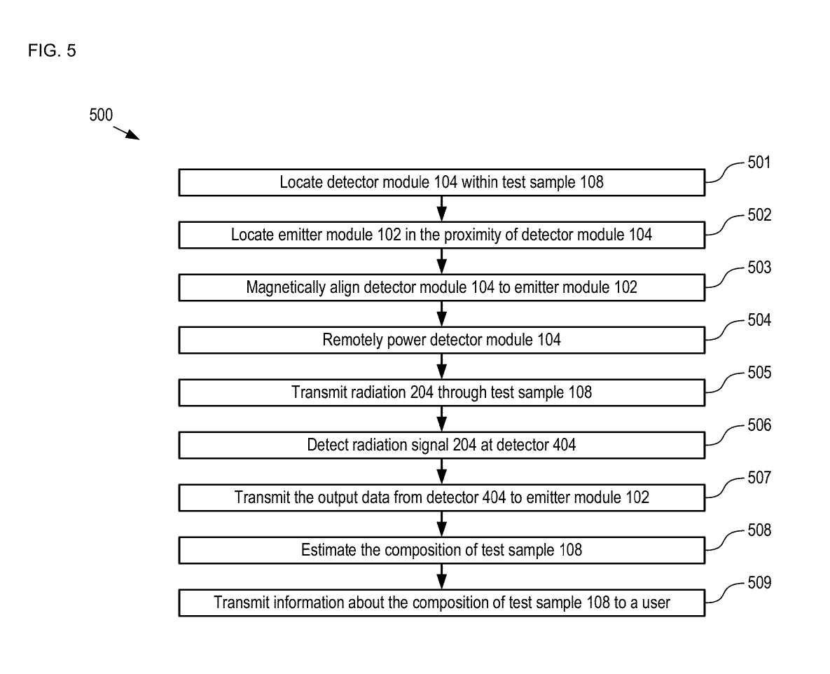

FIG. 5 depicts operations of a method suitable for analyzing a test sample in accordance with the illustrative embodiment. Method 500 begins with operation 501, wherein detector module 104 is implanted into test sample 108. Method 500 is described herein with continuing reference to FIG. 1 through FIGS. 4A-B.

In the depicted example, test sample 108 is a human forearm, into which the detector module is implanted using convention techniques (e.g., surgery, implantation through a catheter, etc.). While system 100 is designed for in-vivo analysis of a human forearm, one skilled in the art will recognize, after reading this Specification, that the present invention is applicable to the analysis of myriad alternative in-vivo test sites on or in a live test subject (human or animal). In some embodiments, emitter module 102 is included as part of a system that can be attached to the test region of interest, such as a watch, wrist band, chest band, back brace, knee brace, cast, soft cast, etc. Further, in some embodiments, system 100 is designed for the analysis of a different type of test sample, such as an ex-vivo sample, such as a fluid, serum, biological tissue, industrial fluid, petroleum product, and the like, which is held in a cuvette or other vessel.

It should be noted that a structure, such as detector module 104, that comprises a biocompatible material (or biocompatible coating) can remain in a body for an indefinite period of time. Further, although a body's defense mechanism tends to give rise to a buildup of a biofilm on foreign material implanted within it, embodiments of the present invention are substantially indifferent to the formation of biofilms on their surfaces due to the nature of MIR spectroscopy.

At operation 502, emitter module 104 is placed in the proximity of the implantation site of the detector module.

At operation 503, detector module 104 is aligned with emitter module 102. As discussed above and with respect to FIGS. 2A-B, to align the detector module with the emitter module a DC current flow is established in coils 206 of alignment features 202-1 and 202-2. These current flows generate magnetic fields that couple into the coils of alignments features 202-3 and 202-4 on detector module 104, thereby inducing current flows in these coils as well. By virtue of the current flow in each of the coils, their respective fiducials become magnetized and create a first magnetic force between alignment features 202-1 and 202-4 and a second magnetic force between alignment features 202-2 and 202-3. These magnetic forces serve to translationally and rotationally register the detector module with emitter module 102, as well as draw the detector module toward the emitter module, thereby reducing the spacing between them.

At operation 504, emitter module 102 wirelessly powers detector module. In operation 504, power system 308 adds an RF signal to the drive current in coil 206 of alignment feature 202-1. This RF signal inductively couples into coil 206 of alignment feature 202-4, which is electrically coupled with power conversion system 428.

In some embodiments, wireless powering of the detector module is done acoustically. In such embodiments, emitter module 102 includes one or more speakers (e.g., piezoelectric transducers) that transmit acoustic energy toward one or more energy scavenging systems (e.g., piezoelectric microphones) located on detector module 104. The energy scavenging systems convert the received acoustic energy into an electrical signal that is then provided to power conversion system 428.

Power conversion system 428 is an electrical circuit operative for rectifying and converting the RF current signal inductively coupled at alignment feature 202-4 into electrical energy that is used to power detector 404 and orchestrate data communications between the emitter and detector modules. Typically, detector module 104 also includes an energy storage system, such as a super capacitor, for storing the converted electrical energy provided by power conversion system 428.

At operation 505, power system 308 energizes filament 318 to generate radiation 204, which is transmitted through test sample 108 to detector module 104. In the depicted example, radiation 204 includes wavelengths that span the range of approximately 2.5 microns to approximately 12.5 microns (i.e., the MIR wavelength range).

In some embodiments, at operation 505, system 100 is operated in a “flash” mode in which radiation 204 is provided as a series of radiation pulses rather than in continuous fashion. As discussed above, the isolation of filament 318 from substrate 302 by cavity 332 reduces its thermal mass, which enables it to respond quickly to changes in its drive current. For example, in some embodiments, filament 318 is energized such that it emits radiation 204 during pulse periods of 10-ms duration during a plurality of 100-ms-long frames; however, duty factors other than 10:1 can be used without departing from the scope of the present invention.

At operation 506, detector elements 410 detect radiation signal 204 after it has passed through test sample 108.

As discussed briefly above, every chemical and chemical compound (i.e., analyte), as well as suspended constituents (e.g., hemoglobin, hematocrit, etc.) in a fluid absorbs radiation at characteristic wavelengths that arise from molecular vibrations based on the nature of the bonds within the analyte molecule. As a result, each constituent in a liquid, such as blood or other bodily fluid, has an absorption spectra that is uniquely characteristic of that constituent.

As discussed in U.S. Pat. Nos. 8,344,323 and 8,541,743, each of which is incorporated herein by reference in their entirety, identifying and quantifying one or more analytes included in a sample comprising a background solvent can be enabled by using the background solvent itself as a wavelength reference.

In some embodiments of the present invention, the spectral signature of the background solvent is substantially removed from a composite spectrum based on the complete chemistry of the sample. As a result, the spectral features associated with constituents in the sample are more easily identified. Further, in some embodiments, the concentration of one or more constituents is estimated by normalizing the spectral characteristics of constituent to the spectral characteristics of the background solvent. In some embodiments, this normalization is enabled by the use of spectral characteristics of the background solvent, itself, to develop an absolute wavelength reference.

The ability to use some background solvents as absolute wavelength references arises from the fact that certain chemicals exhibit temperature invariant behavior that is linked to specific wavelength characteristics. Water is one such chemical. In water, for example, molecular vibrations due to the nature of the bonds within a water molecule, and mediated by the molecular structure, gives water a distinct spectra comprising temperature invariant features. By registering an absorption spectrum measured for a sample solution to these known features, the characteristic absorption spectrum of water can be removed from a measured composite spectrum to isolate and identify analytes and/or other constituents in the sample solution.

At operation 507, communications module 430 transmits the output data from detector 404 to communications circuit 340 via communications module 430.

Communications module 430 transmits the output data to communications circuit 340 via an RF signal transmitted from coil 206 of alignment feature 202-3 to coil 206 of alignment feature 202-2. In the depicted example, the output data is encoded as 8-bit byte words in a pulse-width modulated format. In some embodiments, an array of sources and detectors is used wherein the coil and “cross” eclipse the source array and are lithographically aligned with the corresponding elements on the detector array.

In some embodiments, communications between emitter module 102 and detector module 104 is effected by acoustic transceivers included in each module. Typically, such acoustic transceivers include piezoelectric-based speakers and microphones; however, myriad acoustic actuators and sensors can be used without departing from the scope of the present invention.

Such embodiments exploit the fact that when microphones and speakers are separated by a millimeter or less of tissue only extremely low energies are required to communicate acoustically. As a result, when the emitter and detector modules are positioned close to one another by alignment system 106, data communication between them can be established using an inaudible bi-tone modem (typically using a 300-3,300 Hz bandwidth) to effect a localized, peer-to-peer acoustic communication network that operates at communication rates of up to 1200 baud using, for example, simple binary frequency shift keying (BFSK) or phase-shift keying (PSK) with reduced sideband power. In some embodiments, a UART modem (ASIC or software implemented) is incorporated into one or both of communications module 430 and communications circuit 340.

At operation 508, communications circuit 338 transmits the output data to controller 110 via conventional Bluetooth signaling protocol. In some embodiments, controller 110 relays the data from detector module 104 to external equipment.

In the depicted example, controller 110 is a conventional smart phone. The use of a smart phone for controller 110 enables alternative embodiments wherein communication between emitter module 102 and controller 110 is effected via an acoustic communications link (rather than a Bluetooth, RF, or other electronic wireless protocol), wherein emitter module 102 includes a speaker and microphone for communicating with the controller. Such embodiments derive significant advantages from the use of an acoustic communications link. For example, contemporary smart phones can implement high-definition voice that more than doubles the range of audio frequencies, which enables higher signaling rates. Further, sound is inherently localized and cannot penetrate walls or propagate over long distances in the air. As a result, acoustic communication does not fall under FCC regulations that might require permits or international frequency constraints that are associated with radio communication media (e.g., Bluetooth, WiFi, cellular, ZigBee, RFID, and proprietary systems).

Typically, the human ear can perceive frequencies as low as 20 Hz and as high as 20,000 Hz. To insure that frequency-shift keying (FSK), or phase-shift keying (PSK), modulation is non-audible to humans, therefore, the modulation frequencies must be differentiated on a carrier frequency that is above a human's audible range (above 20,000 Hz for example). Exemplary PSK tones in accordance with the present invention include 21,200 and 22,100 Hz; however, any suitable tone can be used without departing from its scope.

Contemporary smart phones have acoustic broadband microphones and speakers plus digital-signal processing and pentatonic codex processing capacity that collectively enables the implementation of an acoustic modem entirely in software. As a result, a software application alone could enable a localized peer-to-peer acoustic communications link for digital device-to-device communications.

Further, commercially available smart phones typically include built-in microphones having sufficient bandwidth for operation in the 20 KHz range. In addition, such smart phones also normally include speakers capable of radiating audio at frequencies greater than 20,000 Hz. As a result, conventional, commercially available smart phones enable bidirectional ultrasonic communication over a range of several feet that is secure and difficult to intercept.

It should be noted that, while it is described in the context of spectroscopy systems, the principle of the acoustic communications link described herein is applicable to a wide range of other applications. In fact, acoustic communications links in accordance with the present invention are applicable to virtually any localized peer-to-peer communications application for which Bluetooth, ZigBee, short-range cellular, or other proprietary wireless communications protocols are suitable.

At operation 509, controller 110 estimates the composition of test sample 108.

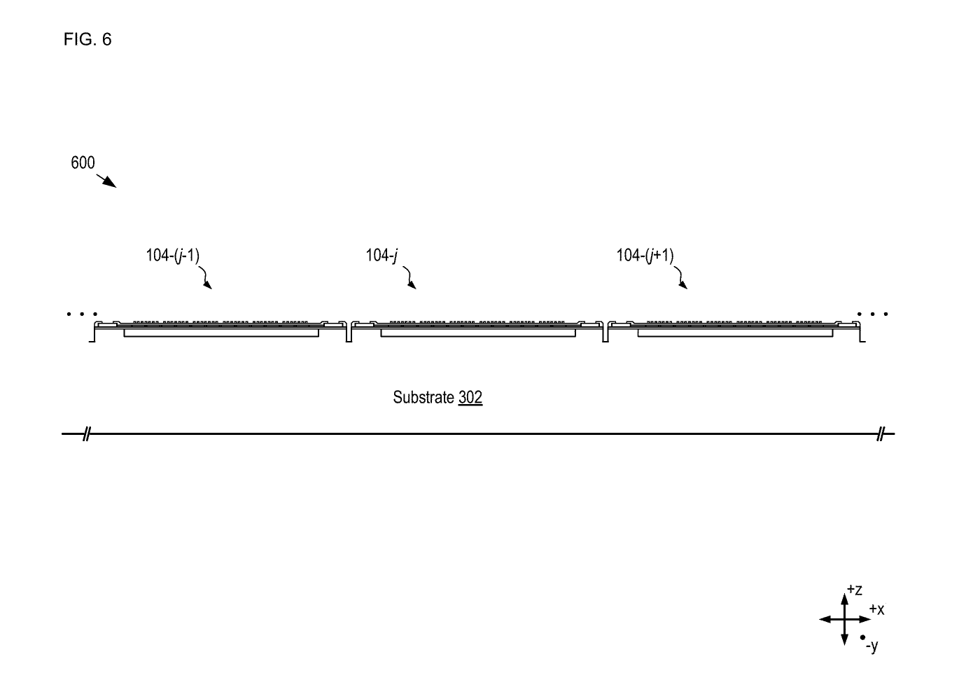

FIG. 6 depicts a radiation source in accordance with an alternative embodiment of the present invention. Source 600 includes an array of source elements 602-1 through 602-M, where M is the number of sub-ranges of wavelengths into which radiation 204 is separated by source 600.

Each source element 602-j, where j=1 through M, is analogous to source 304 described above and with respect to emitter module 102; however, the nanodots, and their pattern, is designed such that its plasmonic resonance restricts the emission of that source element to a different one of the M sub-ranges of wavelengths that collectively define radiation 204. Each sub-range of wavelengths is approximately equal to 1/M of the spectral range of radiation 204. Typically, the ratio of detector elements to source elements (i.e., N:M) is within the range of 1:1 to 100:1.

In some embodiments, each source element 602-j, is optically coupled with a lens that further sharpens the spectral content of its emitted radiation and focuses or collimates that radiation onto a corresponding detector element or elements in detector 404.

Typically, each source element of source 600 is individually addressable, which enables a variety of flash modes to be used for generating radiation 204. These modes range from energizing each source element one at a time through energizing them as a whole. Preferably, however, groups of source elements are energized simultaneously, where the groups can be either contiguous or non-contiguous. In some embodiments, the source elements are energized in groups that are selected to mitigate the excitation of harmonics—for example, in three groups that include: (1) the source elements that emit wavelengths within the range of 2.5 microns to 4.0 microns; (2) the source elements that emit wavelengths within the range of 4.0 microns to 7.0 microns; and (3) the source elements that emit wavelengths within the range of 7.0 to 12.5 microns.

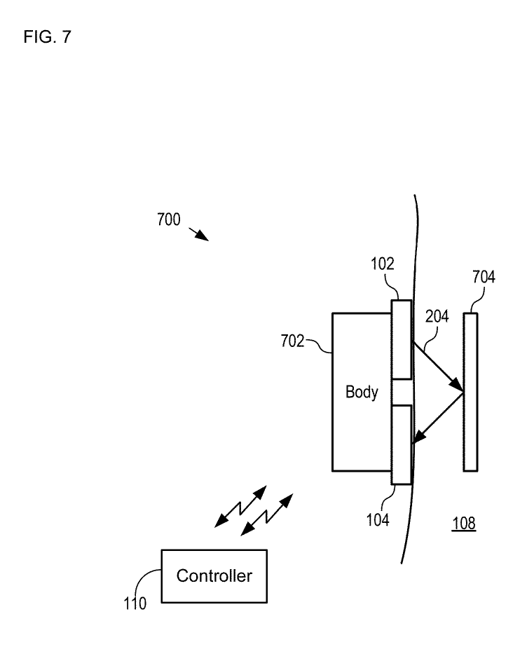

FIG. 7 depicts a spectroscopy system in accordance with an alternative embodiment of the present invention. System 700 includes body 702, mirror 704, emitter module 102, detector module 104, and controller 110.

Body 702 is a structurally rigid mount that locates emitter module 102 and detector module 104.

Mirror 704 is a mechanically rigid element that is reflective for radiation 204.

When mirror 704 is implanted within test sample 108, emitter module 102, detector module 104, and mirror 704 collectively define an optical path through test sample 108 along which radiation 204 propagates. It should be noted that the path length through test sample 108 for system 700 is typically longer than the path length of system 100.

System 700 operates in analogous fashion to system 100.

In some embodiments, emitter module includes a lens for directing radiation 204 at an angle toward mirror 704 to facilitate its receipt by detector module 104.

In some embodiments, system 700 includes alignment system 106 for ensuring the proper alignment and orientation of mirror 704 relative to emitter module 102 and detector module 104.

It is to be understood that the disclosure teaches just one example of the illustrative embodiment and that many variations of the invention can easily be devised by those skilled in the art after reading this disclosure and that the scope of the present invention is to be determined by the following claims.

What is claimed is:

1. A spectroscopy system comprising a plurality of components that includes:
(1) an emitter module comprising a source for providing a first radiation signal having a first spectral range that includes the mid-infrared spectral range;
(2) a detector module comprising a detector that includes a plurality of detector elements, each detector element being selectively sensitive for a different first sub-range of wavelengths within the first spectral range;
wherein, when operatively coupled, the source and detector collectively define an optical path that includes a first region of a test sample; and
wherein a first component of the plurality thereof is dimensioned and arranged to be implanted within the test sample during operation of the spectroscopy system, the optical path including the first component.

2. The spectroscopy system of claim 1 wherein the source includes:
(a) a filament comprising a first material that is electrically conductive;
(b) a first layer disposed on the filament, the first layer comprising a dielectric material; and
(c) second layer disposed on the first layer, the second layer comprising a plurality of features that is arranged in a periodic arrangement;
wherein the filament, first layer and second layer collectively define a plasmonic resonant structure that is operative for providing first radiation signal such that it that has higher intensity within a first spectral range than outside the first spectral range.

3. The spectroscopy system of claim 1, wherein the intensity of the first radiation signal at each wavelength within the first spectral range is at least three orders of magnitude greater than the intensity of the first radiation signal at any wavelength outside the first spectral range.

4. The spectroscopy system of claim 1 wherein the source includes a plurality of source elements, each source element being operative for providing radiation within a different second sub-range of wavelengths such that the plurality of source elements collectively provides the first radiation signal.

5. The spectroscopy system of claim 1 wherein the first component includes the detector module.

6. The spectroscopy system of claim 5 further comprising (3) an alignment system including:
(a) a first alignment feature that includes:
(i) a first coil for generating a first magnetic field; and
(ii) a first fiducial that includes a soft magnetic material;
wherein the first fiducial and the first coil are dimensioned and arranged to enable magnetic coupling between them; and
(b) a second alignment feature that includes:
(i) a second coil for generating a first magnetic field; and
(ii) a second fiducial that includes a soft magnetic material;
wherein the second fiducial and second first coil are dimensioned and arranged to enable magnetic coupling between them;
wherein the first alignment feature and second alignment feature are dimensioned and arranged to generate an attractive force between the first fiducial and second fiducial when a first electric current flows in the first coil.

7. The spectroscopy system of claim 6 wherein the emitter module includes the first alignment feature, and wherein the detector module includes the second alignment feature.

8. The spectroscopy system of claim 7 wherein the emitter module further includes a power circuit, and wherein the first coil and second coil enable wireless transmission of electrical power from the emitter module to the detector module.

9. The spectroscopy system of claim 7 wherein the emitter module further includes a first communications module, and wherein the detector module further includes a second communications module, and further wherein the first and second communications modules are operatively coupled when the emitter module and detector module are operatively coupled.

10. The spectroscopy system of claim 5 wherein the emitter module further includes an acoustic transmitter, and wherein the detector module further includes an energy-scavenging system that is operative for converting acoustic energy into electrical energy.

11. The spectroscopy system of claim 1 wherein the plurality of components includes (3) a mirror for reflecting the first radiation signal, wherein the first component includes the mirror.

12. The spectroscopy system of claim 11 further comprising an alignment system including:
(a) a first alignment feature that includes:
(i) a first coil for generating a first magnetic field; and
(ii) a first fiducial that includes a soft magnetic material;
wherein the first fiducial and the first coil are dimensioned and arranged to enable magnetic coupling between them; and
(b) a second alignment feature that includes:
(i) a second coil for generating a first magnetic field; and
(ii) a second fiducial that includes a soft magnetic material;
wherein the second fiducial and second first coil are dimensioned and arranged to enable magnetic coupling between them;
wherein the first alignment feature and second alignment feature are dimensioned and arranged to generate an attractive force between the first fiducial and second fiducial when a first electric current flows in the first coil; and
wherein the emitter module includes the first alignment feature and the mirror includes the second alignment feature.

13. The spectroscopy system of claim 1 wherein the second layer has a top surface and a bottom surface, and wherein each feature of the plurality thereof extends from the top surface to the bottom surface.

14. The spectroscopy system of claim 1 wherein each feature of the plurality thereof has a cross-sectional shape that is dimensioned and arranged to give rise to a characteristic resonant frequency for each feature.

15. The spectroscopy system of claim 1 wherein each feature of the plurality thereof is a projection having a first thickness.

16. The spectroscopy system of claim 1 further comprising a cavity, wherein at least a portion of the cavity is between the plasmonic resonator structure and a first substrate on which the source is disposed.

17. The spectroscopy system of claim 1 wherein the detector is dimensioned and arranged such that each detector element of the plurality thereof includes:
(a) a thermal transducer; and
(b) an absorber comprising a photonic crystal;
wherein the absorber and the thermal transducer are thermally coupled;
wherein each photonic crystal of the plurality thereof is dimensioned and arranged to enable its respective absorber to selectively absorb light within a different first sub-range of wavelengths of the plurality thereof.

18. The spectroscopy system of claim 1 wherein the emitter is operative for communicating with a controller via an RF communications link.

19. The spectroscopy system of claim 1 wherein the emitter is operative for communicating with a controller via an acoustic communications link.

20. A method for analyzing a test sample, the method comprising:
providing a spectroscopy system that includes a plurality of components that comprises an emitter module having a source for generating a first radiation signal characterized by a first spectral range that includes the mid-infrared spectral range;
locating a first component of the plurality thereof within the test sample;
transmitting the first radiation signal along a first optical path through a first region of the test sample, the first optical path including the first component;
detecting a second radiation signal that includes at least a portion of the first radiation signal after it has passed through the first region; and;
estimating the composition of the test sample based on absorption of the first radiation signal in the first region.

21. The method of claim 20 further comprising providing the emitter module such that the source includes a plasmonic resonant structure that is dimensioned and arranged to generate the first radiation signal such that the first radiation signal has higher intensity within the mid-infrared spectral range than outside the mid-infrared spectral range.

22. The method of claim 20 wherein the second radiation signal is detected at a detector module comprising a plurality of detector elements, and wherein each detector element includes:
a thermal transducer; and
an absorber comprising a photonic crystal, the absorber being thermally coupled with the thermal transducer;
wherein each photonic crystal of the plurality thereof is dimensioned and arranged to enable its respective absorber to selectively absorb light within a different first sub-range of wavelengths within the first spectral range.

23. The method of claim 22 further comprising locating the emitter module outside the test sample, wherein the first component includes the detector module.

24. The method of claim 23 further comprising aligning the emitter module and the detector module.

25. The method of claim 24 wherein the emitter module and detector module are aligned by generating a magnetic force between a first alignment feature in the emitter module and a second alignment feature in the detector module.

26. The method of claim 25 wherein the magnetic force is generated by operations comprising:
generating a first current flow in a first coil that is magnetically coupled with a first fiducial, the first coil and first fiducial being included in the first alignment feature; and
enabling the first current flow to induce a second current flow in a second coil that is magnetically coupled with a second fiducial, the second coil and second fiducial being included in the second alignment feature.

27. The method of claim 26 further comprising powering the detector module by operations comprising:
generating a first alternating current in the first coil;
enabling inductive coupling between the first coil and second coil, wherein the inductive coupling gives rise to a second alternating current in the second coil; and
converting the second alternating current into electrical energy.

28. The method of claim 23 further comprising powering the detector module by operations comprising:
generating acoustic energy at the emitter module; and
converting the acoustic energy into electrical energy at the detector module.

29. The method of claim 23 further comprising establishing a communications link between the emitter module and the detector module.

30. The method of claim 29 wherein the communications link is established as a radio-frequency link.

31. The method of claim 30 further comprising transmitting a first communication signal between the emitter module and the detector module, wherein the first communication signal is transmitted by operations comprising:
generating a first RF signal in one of the first coil and second coil;
enabling inductive coupling between the first coil and second coil; and
detecting a second RF signal in the other one of the first coil and second coil, wherein the second RF signal is based on the first RF signal.

32. The method of claim 29 wherein the communications link is established as an acoustic link.

33. The method of claim 20 wherein the first component comprises a mirror that is operative for reflecting the first radiation signal, wherein the first optical path includes the mirror.

34. The method of claim 20 further comprising providing the emitter module such that the source includes a plurality of source elements, each source element being operative for providing radiation within a different second sub-range of wavelengths such that the plurality of source elements collectively provides the first radiation signal.

35. The method of claim 34 wherein the plurality of source elements includes a plurality of source-element groups, and wherein the source is energized by serially selectively energizing each source-element group of the plurality thereof.

36. The method of claim 35 wherein the source is energized such that the plurality of source-element groups includes:
a first source-element group that collectively emits radiation that spans the wavelength range from 2.5 microns to 4.0 microns;
a second source-element group that collectively emits radiation that spans the wavelength range from 4.0 microns to 7.0 microns; and
a third source-element group that collectively emits radiation that spans the wavelength range from 7.0 microns to 12.5 microns.

37. The method of claim 20 further comprising determining the presence of a first constituent in the test sample, wherein the first constituent is one of a solution-based constituent and a constituent that is in suspension.

38. The method of claim 37 further comprising determining the concentration of the first constituent in the test sample.

39. The method of claim 20 wherein the source is energized by providing it with a periodic drive signal comprising a series of drive pulses.

40. The method of claim 39 wherein the periodic drive signal has a duty factor that is less than or equal to 50%.

41. The method of claim 20 further comprising establishing a communications link between the emitter module and a controller, wherein the estimate of the composition of the test sample is generated at the controller.

42. The method of claim 41 wherein the communications link is established as a radio-frequency link.

43. The method of claim 41 wherein the communications link is established as an acoustic link.
